(19) United States
(12) Plant Patent Application Publication
(10) Pub. No.: US 2002/0092050 P1
(43) Pub. Date: Jul. 11, 2002

(54) Hybrid tea rose plant named 'Wezgrey'

(76) Inventor: O. L. Weeks, Chino, CA (US)

Correspondence Address: BURNS DOANE SWECKER & MATHIS L L P, POST OFFICE BOX 1404, ALEXANDRIA, VA, 22313-1404, US

(21) Appl. No.: 09/756,731

(22) Filed: Jan. 10, 2001

(51) Int. Cl. A01H 5/00

(52) U.S. Cl. PLT/132

(57) ABSTRACT

A new and distinct variety of Hybrid Tea rose plant is provided which forms attractive double blossoms that are a blend of soft white and lavender. The plant exhibits an upright, vigorous and uniform growth habit, dense medium green foliage, and good disease resistance. The attractive medium green foliage contrasts nicely with the light-colored blossoms. The new plant growth commonly assumes a burgundy appearance. The new variety is particularly well suited for growing as attractive ornamentation in the landscape such as in parks and gardens. Cut flower production also is possible.

### SUMMARY OF THE INVENTION

The new variety of Rosa hybrida Hybrid Tea rose plant was created by artificial pollination wherein two parents were crossed which previously had been studied in the hope that they would contribute the desired characteristics. The female parent (ie, the seed parent) of the new variety was the ‘Silver Spoon’ variety (non-patented in the United States). The male parent (ie, the pollen parent) was the ‘Paradise’ variety (U.S. Plant Pat. No. 4,552). The parentage of the new variety can be summarized as follows:

‘Silver Spoon’×‘Paradise’.

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:

(a) exhibits an upright, vigorous and uniform growth habit,

(b) forms attractive double blossoms that are a blend of soft white and lavender,

(c) forms dense medium green semi-glossy foliage that contrasts well with the light-colored blossoms,

(d) exhibits good disease resistance, and

(e) is particularly well suited for growing as attractive ornamentation.

The new variety well meets the needs of the horticultural industry and can be grown to advantage for cut flower production, as a specimen plant, or in a mass planting.

The new variety has been found to undergo asexual propagation by a number of routes, including budding, grafting, and cuttage. Asexual propagation by the above-mentioned techniques at Wasco, Calif. has shown that the characteristics of the new variety are stable and are strictly transmissible by such asexual propagation from one generation to another.

The new variety has been named the ‘Wezgrey’ variety. The new variety is being marketed under the WORLD WAR II MEMORIAL ROSE trademark.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were three years of age and were observed during the fall while growing outdoors on their own roots at Wasco, Calif. Dimensions in inches and centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem with foliage;

FIG. 13—illustrates a specimen of a main branch with foliage;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of maturing petals.

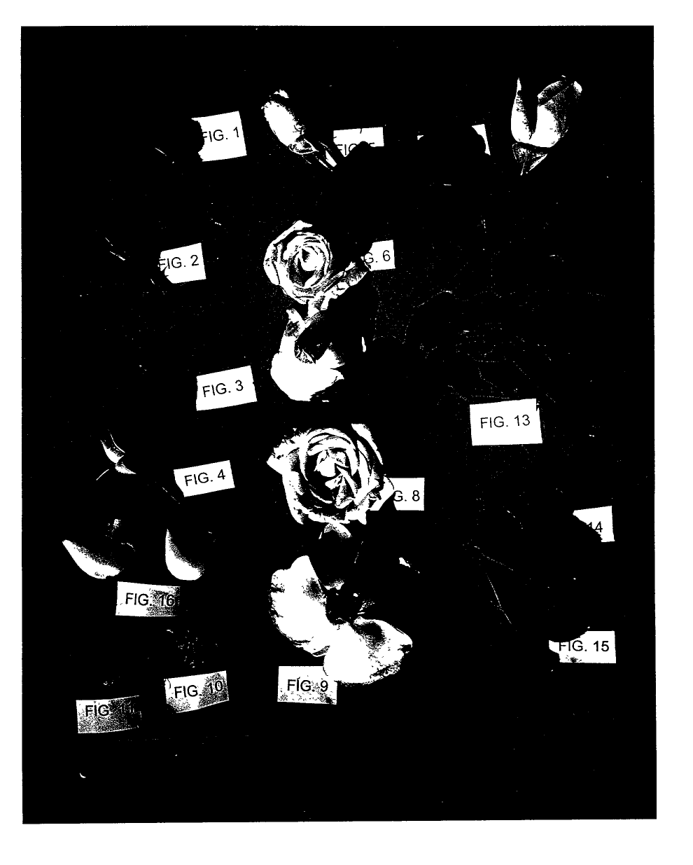

### DETAILED DESCRIPTION

The chart used in the identification of the colors is that of The Royal Horticultural Society (R.H.S. Colour Chart). The description is based on the observation of two year-old plants during October while growing on their own roots outdoors at Wasco, Calif. Color terminology is to be accorded its ordinary dictionary significance.

Class: Hybrid Tea.

Plant:

Height.—approximately 4 feet at the end of the growing season.

Width.—approximately 2 feet at the end of the growing season.

Habit.—upright.

Branches:

Color.—young stems: Yellow-Green Group 144C with highlights of Red Group 53B. adult wood: Yellow-Green Group 146B.

Thorns.—size: approximately 5 mm in length on average. quantity: moderately numerous, approximately 15 true thorns plus bristles on a stem having a length of 30 cm. color: near Greyed-Orange Group 185B. shape: needle-shaped as well as some bristles.

Leaves:

Stipules.—narrow, triangular, and with auricle facing outwards.

Petioles.—upper surface: Yellow-Green Group 144C. under surface: Yellow-Green Group 144A.

Leaflets.—number: 3, 5, and 7. shape: ovate with serrate margins, rounded base and acute apices. serration: regular. texture: consistent. general appearance: dense, leathery, medium green, and with a semi-glossy finish. color (young foliage): upper surface: Green Group 141B to 141C with highlights of Greyed-Purple Group 184B. The new plant growth commonly assumes a burgundy appearance. under surface: Green Group 141C with highlights of Greyed-Purple Group 184B. color (adult foliage): upper surface: Green Group 137A. under surface: Yellow-Green Group 146B.

Inflorescence:

Number of flowers.—usually one to three flowers per stem.

Peduncle.—Yellow-Green Group 144B with prickles near Greyed-Purple Group 185B, and the length is approximately 6 cm on average.

Sepals.—upper surface: near Yellow-Green Group 144C with pubescence of White Group 155D. under surface: Yellow-Green Group 144C with some extensions.

Buds.—shape: pointed. size: large. length: approximately 3 cm on average. color upon opening: upper surface: Yellow-Green Group 150D with highlights of Purple Group 75D. under surface: Yellow-Green Group 150D.

Flower.—shape: double and high-centered. diameter: approximately 7 cm on average. color (when opening begins): upper surface: near White Group 155A and 155B. under surface: combination of Red Group 56D and White Group 155A. color (when blooming): upper surface: near White Group 155A. under surface: near White Group 155D. color (at end of opening): upper surface: near White Group 155A. under surface: near White Group 155D. fragrance: present. petal shape: orbicular. petal number: approximately 25 on average. stamen number: approximately 85 on average. anthers: near Yellow-Orange Group 16B in coloration. filaments: near Yellow-White Group 158B in coloration. pistals: approx imately 40 on average. stigmas: near Yellow-Orange Group 18B in coloration. styles: near Yellow-Orange Group 18A in coloration. receptacle: separate and free with achenes on the bottom and the wall.

Development:

Vegetation.—vigorous and uniform.

Blooming.—mostly single blooms and sometimes in clusters.

Resistance to diseases.—good.

I claim:

1. A new and distinct variety of Hybrid Tea rose plant characterized by the following combination of characteristics:
(a) exhibits an upright, vigorous and uniform growth habit,
(b) forms attractive double blossoms that are a blend of soft white and lavender,
(c) forms dense medium green semi-glossy foliage that contrasts well with the light-colored blossoms,
(d) exhibits good disease resistance, and
(e) is particularly well suited for growing as attractive ornamentation;
substantially as herein shown and described.
